(19) United States
(12) Patent Application Publication
(10) Pub. No.: US 2023/0061572 A1
(43) Pub. Date: Mar. 2, 2023

(54) AUTOMATIC SYSTEM TO MONITOR HEALTH CONDITION OF AN INFANT

(71) Applicant: Mirza Faizan, Irving, TX (US)

(72) Inventors: Mirza Faizan, Irving, TX (US); Akram Yosuf Ibrahim, Garland, TX (US); Inayah Iftikhar, McKinney, TX (US); Zainab Kashif, McKinney, TX (US); Afreen Haq, Arlington, TX (US); Affan Haq, Arlington, TX (US); Omar Abdul Rahiman, Parker, TX (US); Mirza Rizwan, Patna (IN); Abdul Muhaimin Taqui, Plano, TX (US); Saadia Asaf, Aligarh (IN); Mansoor Hasan Khan, Aligarh (IN)

(21) Appl. No.: 17/460,179

(22) Filed: Aug. 28, 2021

(51) Int. Cl. A61B 5/0205; A61B 5/00

(52) U.S. Cl. CPC A61B 5/02055; A61B 5/6892; A61B 5/746; A61B 5/7275; A61B 2562/066; A61B 2562/0247; A61B 2562/0271; A61B 2503/045

(57) ABSTRACT

The present invention provides an automatic system (10) to monitor health condition of an infant (40), said system (10) comprises: a mattress (20) configured to receive the infant (40) in a resting position; a plurality of sensors (30), connected to the mattress (20), to detect the health condition of the infant (40) resting on the mattress (20); a processing module (50), connected to the plurality of sensors (30), to receive information on the health condition of the infant (40) from the plurality of sensors (30) and process the information according to pre-determined criteria; and an output module connected to the processing module to output the processed information.

### FIELD OF THE INVENTION

The present invention relates to an automatic system to monitor health condition of an infant.

### BACKGROUND OF THE INVENTION

Sudden infant death syndrome (SIDS) is when a seemingly healthy baby dies unexpectedly and suddenly, and there is no explanation for the cause of their death. Even though SIDS is considered rare, it's the most common cause of death for children between the age of 1 month and 1 year. It most often happens between the ages of 2 and 4 months. In 2018, approximately 1,300 babies died of SIDS in the United States.

Many of these risk factors causing death of the infants due to SIDS can be avoided. The most significant risk factor: laying your baby to sleep on their stomach or side before the age of 1, co-sleeping—sharing a bed with a parent or caregiver, unsafe or old crib, bedding or mattress that's too soft, crib that contains soft objects to name a few. Avoiding as many of these risk factors as possible will reduce the baby's risk of SIDS.

Conventionally, in order to overcome the issue of SIDS, it is known to the baby close to you when they are sleeping in the same room, but not in the same bed. Other measures include, but not limited to, avoiding co-sleeping (bed sharing) with your baby or letting them sleep with other children or adults, remove toys, bumper pads, blankets, sleep positioners, and pillows from the crib when putting the baby down to sleep, avoid overwrapping (swaddling) the baby when putting them down to sleep and use of a safety-approved crib mattress and place a fitted sheet over it. However, all these measures are basic human measures, which are prone to defects due to the lack of automation and maximum surveillance. Thus, there is a need to have baby monitors and devices to reduce the risk of SIDS.

In order to overcome these drawbacks, prior art suggest a system including a monitoring device dimensioned for placement on a human. The monitoring device including a plurality of sensors for monitoring a sleep position, a temperature, a carbon dioxide level and a respiration of the human. The system further including a processing unit coupled to the monitoring device, the processing unit capable of processing information from the plurality of sensors and performing an algorithm to determine the presence of a sudden infant death syndrome (SIDS) risk factor based on the information. A method may include monitoring two or more of a sleep position, a respiration, a temperature and a carbon dioxide level of a subject. An algorithm may be automatically performed based on the monitoring, to determine the presence of a SIDS risk factor. The results of the algorithm may be displayed and an alert can be sent to, for example, a mobile device based on the risk level.

Another prior art suggests a portable monitoring device for monitoring of the short term, mid term and long term risks of Sudden Infant Death Syndrome (SIDS) and to estimate the risk of onset of positional plagiocephaly. The monitoring device acquires one or more signals associated to the typical breath and positioning patterns of an infant through a tri-axial accelerometer, and registers the data in electronic format, where statistical analysis of the signal and pattern recognition are performed in real time by a processing unit. The device monitors the breath activity of an infant in various conditions, alerting a caregiver when a potentially dangerous situation is detected. The monitoring is attached to the clothes of the infant. Statistical analysis of the registered data is performed for quantifying the long-term SIDS risk, the risk of plagiocephaly and for optimizing the functional parameters of the monitoring device.

Yet another prior art suggest personal warning apparatus comprising at least one relative motion detector adapted to be positioned on or around the body of an individual. The apparatus further comprising an evaluation module having a predetermined activation threshold and at least one communication means. The at least one relative motion detector being operatively linked to the evaluation module, and the evaluation module being operatively linked to the at least one communication means. The evaluation module is configured to be responsive to an evaluation and is configured to send an alarm signal to the at least one communication means. The relative motion detector is adapted to detect lateral movement where lateral movement is in the direction of the longest axis of the individual's body.

It is also commonly known to have a device for reducing the possibility of sudden infant death syndrome (SIDS). The device comprises a position-indicating device effectively coupled to a signal-producing circuit and attached to the clothing of the infant. The position-indicating device provides signals varying in response to prone and other positions assumed by the infant during sleep, allowing an associated alarm device to be activated in response to the infant's assuming a SIDS-dangerous prone or side-lying position. It is known to have the position of the infant can be determined by an optical sensor interacting with a reflective or other marker adhered to the infant. Gravity or pressure switches may also be used to provide position-responsive signals. A signal generated upon assumption of the SIDS-dangerous prone or side-lying positions is transmitted to a remote receiver located proximate the infant's care-giver whereupon an alarm is generated to indicate the need to reposition the infant. A constant low-level or intermittent maintenance signal can be produced to assure the continued and proper operation of the apparatus. An additional awakening alarm can be produced near the sleeping infant to further reduce the likelihood of a SIDS event.

However, the above-cited prior arts have drawbacks. The known devices to track SIDS lack capability to accurately track the infant's health condition and to measure the weight distribution for accurate detection of health condition. Thus, the known devices to track SIDS are prone with errors to inaccurately detect the resting position of the infants and produce fault alarms. Accordingly, there is a need to provide a solution to overcome all these drawbacks of prior art.

The drawbacks of prior art are overcome by the present invention, as detailed in the forthcoming sections of the present application.

### OBJECTS OF THE INVENTION

The main object of the present invention is to provide an automatic system to monitor health condition of an infant.

Another object of the present invention is to provide an automatic system to monitor health condition of an infant, which accurately detects the health conditions of the infants.

Another object of the present invention is to detect the accurate health conditions of the infants and to reduce the possibility of false alarms.

Another object of the present invention is to detect the actual resting position of the infant.

Another object of the present invention is to accurately detect the extra weight and pressure applied on the infant's body.

Another object of the present invention is to detect which part of the infant's body has extra weight/pressure.

### SUMMARY OF THE INVENTION

In order to solve the drawbacks of the prior art, the present invention provides an automatic system to monitor health condition of an infant

According to an embodiment of the present invention, an automatic system to monitor health condition of an infant is disclosed. The system comprises a mattress configured to receive the infant in a resting position. Thus system further comprises a plurality of sensors, connected to the mattress, to detect the health condition of the infant resting on the mattress. Thus system further comprises a processing module, connected to the plurality of sensors, to receive information on the health condition of the infant from the plurality of sensors and process the information according to pre-determined criteria. Thus system further comprises an output module connected to the processing module to output the processed information.

In some embodiments, the plurality of sensors are embedded in the mattress forming a matrix of sensors.

In some embodiments, the plurality of sensors comprise sensors to detect weight of the infant, resting position of the infant, weight of all the body parts of the infant, and all other health conditions.

In some embodiments, the matrix of the plurality of sensors is configured to measure the weight of each part of the infant, like an upper part of the body, i.e. Head, Chest, Abdomen etc, and/or a lower part of the body (legs).

In some embodiments, the processing module is configured to detect a side of the mattress where the head of the infant is placed and/or to detect another side of the mattress where the legs of the infant are placed.

In some embodiments, the processing module is configured to generate an alarm if the weight of the infant's body part resting on ether sides of the mattress increase more than the predetermined criteria.

In some embodiments, the processing module is further configured to generate a report of an amount of extra weight placed on the infant's body part resting on ether sides of the mattress.

In some embodiments, the automatic system to monitor health condition of an infant further comprises at least one smart camera focused on the mattress to detect the resting position of the infant.

In some embodiments, the smart camera monitors the resting position of the infant.

In some embodiments, the processing module is further configured to process the health information from the smart cameras and the plurality of sensors.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a perspective view of an automated system having a mattress embedded with a plurality of sensors to carry an infant in the resting position according to an embodiment of the present invention.

FIG. 2 is a block diagram of an automated system according to an embodiment of the present invention.

Persons skilled in the art will appreciate that elements in the figures are illustrated for simplicity and clarity and may not have been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF DRAWINGS

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

Embodiments of the present invention will be described below in detail with reference to the accompanying drawings.

It will be understood by those skilled in the art that the foregoing general description and the following detailed description are explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a nonexclusive inclusion, such that a process or method that comprises a list of steps does not include only those steps but may include other steps not expressly listed or inherent to such process or method. Similarly, one or more devices or subsystems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfil the requirements of uniqueness, utility and non-obviousness.

Embodiments of the present invention will be described below in detail with reference to the accompanying drawings.

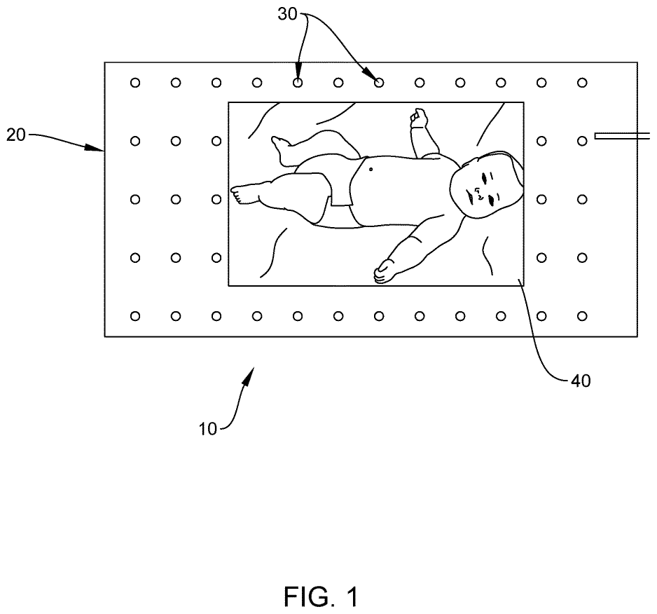

FIG. 1 is a perspective view of an automated system (10) having a mattress (20) embedded with a plurality of sensors (30) to carry an infant (40) in the resting position according to an embodiment of the present invention. In some embodiments, the automatic system (10) to monitor health condition of an infant (40) comprises a mattress (20). In some embodiments, the mattress (20) may be configured to receive the infant (40) in a resting position. In some embodiments, the mattress (20) may be placed in a bed or a cot of the infant. The mattress (20) may be formed of any conventional material, like foam etc. that is comfortable for the infant.

According to an embodiment of the present invention, the automatic system (10) to monitor health condition of an infant (40) further comprises a plurality of sensors (30), connected to the mattress (20), to detect the health condition of the infant (40) resting on the mattress (20). In some embodiments, the plurality of sensors (30) may comprise of any electronic, digital, analogue or any other known sensors for measuring the health conditions of the infant. In particular, the plurality of sensors (30) may have sensors to detect at least the parameter of weight, pressure, temperature, physiological sensors, ECG sensor, heart monitoring sensor, oximeter, piezoelectric elements, thermometers, insulin detectors, blood-oxygen sensor, impedance for plethysmography, carotid pulse wave detector(s) to name a few. In some embodiments, the plurality of sensors (30) comprise sensors to detect weight of the infant, pressure sensors, temperature sensors, oxygen monitoring, resting positions of the infant, weight and/or pressure of all the body parts of the infant, and all other health conditions.

In some embodiments, the plurality of sensors (30) may be comprised in a sensor sheet placed inside, outside and/or underneath the mattress (20). In some embodiments, the plurality of sensors (30) may be arranged directly in the mattress (30) or on the sensor sheet such that the arrangement of sensors (30) forms a matrix of the plurality of sensors (30).). In some embodiments, the plurality of sensors (30) are embedded in the mattress (20) forming a matrix of sensors.

In some embodiments, the matrix of the plurality of sensors (30) may be configured to adopt the system for measuring health conditions according to weight distribution of the infant's body. In some embodiments, matrix measures the weight of each part of the infant, like an upper part of the body, i.e. Head, Chest, Abdomen etc., and/or a lower part of the body (legs). At least one effect can be that the system (10) is made capable of accurately measuring weight, pressure and other physiological conditions of each part of the infant's body accurately.

In some embodiments, the plurality of sensors (30) is configured to detect a side of the mattress (20) where the head of the infant (40) is placed and/or to detect another side of the mattress (20) where the legs of the infant (40) are placed. At least one effect can be that the system (10) can immediately send alerts in case of any emergency depending on which side of the mattress (20) infant has placed the head or legs.

According to an embodiment of the present invention, the automatic system (10) to monitor health condition of an infant (40) further comprises a processing module. In some embodiments, the processing module is connected to the plurality of sensors (30), to receive information on the health condition of the infant (40) from the plurality of sensors (30) and process the information according to pre-determined criteria. In some embodiments, the sensors (30) read information about all the health conditions of the infant (40) and send that data to the processing module. In some embodiments, the processing module, even if the sensors don't detect, is configured to detect a side of the mattress (20) where the head of the infant (40) is placed and/or to detect another side of the mattress (20) where the legs of the infant (40) are placed.

In some embodiments, the processing module is configured to generate an alarm if the weight of the infant's body part resting on ether sides of the mattress increase more than the predetermined criteria. In some embodiments, the alarm is sent directly to the parents via network. The alarm may be sent in the form of a call, or a message. In some embodiments, the alarm may be sent through a ringing bell placed in the parent's room or in the vicinity of the parents' presence. In some embodiments, the processing module is further configured to generate a report of an amount of extra weight placed on the infant's body part resting on ether sides of the mattress (20). In some embodiments, the report may be sent directly to the parents' mobile phone through message or email. In some embodiments, the processing module is configured to generate an alarm if the weight of the infant's body part resting on ether sides of the mattress increase more than the predetermined criteria.

According to an embodiment of the invention, the system (10) may further comprise a storage module (not shown). In some embodiments, the predetermined criteria may comprise of standard weight of the infants stored in the system for processing the health conditions of the infant. In some embodiments, the predetermined criteria may comprise of all standard physiological conditions stored in the storage module. The physiological conditions may be categorised in the system (10) or in the storage module on the basis of the age of the infants, like one month old, two months old and so on. This enables the system to have accurate processing of the health conditions and reduce chances of generating false alarms. Thus, any extra weight or pressure on the mattress (20) would be compensated by the standard parameters stored in the storage module.

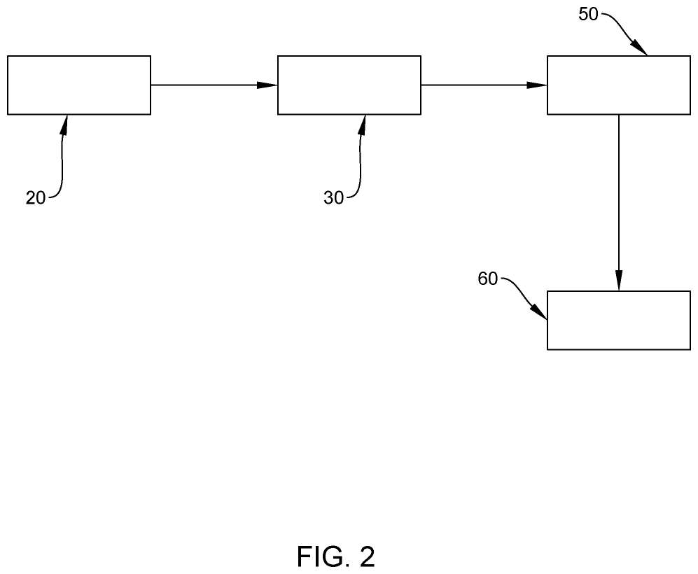

According to an embodiment of the present invention, the automatic system to monitor health condition of an infant further comprises an output module (60) connected to the processing module to output the processed information, as shown in FIG. 2. The output module may be any electronic device connected to the parents' mobile phones, computers, etc. to provide immediate information about the infant's health conditions.

According to an embodiment of the present invention, the automatic system to monitor health condition of an infant further comprises at least one smart camera focused on the mattress (20) to detect the resting position of the infant. In some embodiments, the system may comprise a plurality of cameras to capture all changes in the resting positions of the infant. There invention could implement any known camera device, which is able to clearly capture the infant. At least one effect can be that the smart camera continuously monitors the resting position of the infant.

According to an embodiment of the present invention, the processing module (50) is further configured to process the health information from the smart cameras and the plurality of sensors (30). At least one effect can be that a complete picture of the infant's sleep is created. In particular, if there is any chance of miscalculations by the sensors, the smart cameras can give a cross confirmation about the accurate infant's resting position. Thus, it reduces the possibility of generating any false alarms.

Moreover, the actions of any components in the block diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the present subject matter, any limitations arising on account thereto, are not intended. As would be apparent to a person in the art, various working modifications may be made to the method in order to implement the inventive concept as taught herein. The drawings and the foregoing description give examples of embodiments.

We claim:

1. An automatic system (10) to monitor health condition of an infant (40), said system (10) comprises:
a. a mattress (20) configured to receive the infant (40) in a resting position;
b. a plurality of sensors (30), connected to the mattress (20), to detect the health condition of the infant (40) resting on the mattress (20);
c. a processing module (50), connected to the plurality of sensors (30), to receive information on the health condition of the infant (40) from the plurality of sensors (30) and process the information according to pre-determined criteria; and
d. an output module (60) connected to the processing module to output the processed information.

2. The automatic system (10) to monitor health condition of an infant (40) as claimed in claim 1, wherein the plurality of sensors (30) are embedded in the mattress (20) forming a matrix of sensors.

3. The automatic system (10) to monitor health condition of an infant (40) as claimed in claim 1, wherein the plurality of sensors (30) comprise sensors to detect weight of the infant, pressure sensors, temperature sensors, oxygen monitoring, resting positions of the infant, weight and/or pressure of all the body parts of the infant, and all other health conditions.

4. The automatic system (10) to monitor health condition of an infant (40) as claimed in claim 3, wherein the matrix of the plurality of sensors (30) is configured with weight distribution mechanism to measure the weight of each part of the infant, like an upper part of the body, i.e. Head, Chest, Abdomen etc, and/or a lower part of the body (legs).

5. The automatic system (10) to monitor health condition of an infant (40) as claimed in claim 4, wherein the processing module (50) is configured to detect a side of the mattress (20) where the head of the infant (40) is placed and/or to detect another side of the mattress (20) where the legs of the infant (40) are placed.

6. The automatic system (10) to monitor health condition of an infant (40) as claimed in claim 5, wherein the processing module (50) is configured to generate an alarm if the weight of the infant's body part resting on ether sides of the mattress increase more than the predetermined criteria.

7. The automatic system (10) to monitor health condition of an infant (40) as claimed in claim 6, wherein the processing module (50) is further configured to generate a report of an amount of extra weight placed on the infant's body part resting on ether sides of the mattress (20).

8. The automatic system (10) to monitor health condition of an infant (40) as claimed in claim 1 further comprises at least one smart camera focused on the mattress (20) to detect the resting position of the infant (40).

9. The automatic system (10) to monitor health condition of an infant (40) as claimed in claim 8, wherein the smart camera monitors the resting position of the infant (40).

10. The automatic system (10) to monitor health condition of an infant (40) as claimed in claim 9, wherein the processing module (50) is further configured to process the health information from the smart cameras and the plurality of sensors (30).
